(19) United States
(12) Patent Application Publication
(10) Pub. No.: US 2012/0283588 A1
(43) Pub. Date: Nov. 8, 2012

(54) ADHESIVE MAGNETIC SYSTEM

(76) Inventors: Jonathan Lowy, Bellingham, WA (US); Leasa Lowy, Bellingham, WA (US)

(21) Appl. No.: 13/098,809

(22) Filed: May 2, 2011

(51) Int. Cl. A61B 5/0444; A61N 1/362; A61N 1/39

(52) U.S. Cl. 600/511; 607/5; 607/36

(57) ABSTRACT

An adhesive magnetic system for modifying an implantable device is provided. The system may include at least one magnet, an enclosure configured to receive the magnet, and an adhesive layer positioned on the enclosure for adhering the enclosure to a patient.

### FIELD

The present invention is directed to an adhesive magnetic system for modifying an implantable device, and in particular to an adhesive magnetic system that is used to activate a magnetic switch or sensor in a device implanted in a patient.

### BACKGROUND

Pacemakers are implantable devices that provide electrical impulses, thereby causing cardiac stimulation when intrinsic myocardial electrical activity is either slow or absent. Implantable Cardioverter Defibrillators (ICDs) are used to treat life threatening abnormal cardiac tachydysrhythmia, and if the heart beats too quickly, the ICD provides an internal electrical shock to restore the heart's normal rhythm.

Implantable devices, such as pacemakers and ICDs, are designed to sense the intrinsic electrical activity of the heart. This makes them sensitive to electromagnetic interference (EMI) exposure. EMI may be external (such as from electrocautery tools, which are commonly used in a variety of medical procedures) or a consequence of pacemaker or ICD malfunction. Exposure to EMI may result in unintended and potentially deleterious consequences to the patient. EMI may cause the device to malfunction which for a pacemaker, could result in the failure to deliver appropriate therapy, and for an ICD, could result in the inappropriate delivery of shock therapy.

Modification of these implantable devices is typically required to prevent EMI exposure during a medical procedure that involves EMI, such as electrocautery. The devices are generally modified by reprogramming the device. Modification or reprogramming of the device may also be required in an unanticipated situation, such as in the event of acute device malfunction. Modification or reprogramming may also be required during the end of a patient's life when pacemaker or ICD therapy is no longer desired by the patient.

Modification can be achieved by utilizing a programming computer manufactured by the implantable device manufacturer. However, these computers require special skill and training, and are not available in all environments and settings where patients with these devices may require reprogramming. Even when the programming computer is available there is not always an appropriately trained individual to operate it.

To provide an alternative to the programming computer, most if not all pacemaker and ICD manufacturers include a system to provide temporary modification or reprogramming without the programming computer. In particular, these devices typically include a magnetic switch or sensor that is activated by a magnetic field to modify the device. The device may go into a temporary reprogramming mode. In most, if not all, current pacemakers and ICDs, the magnetic switch is a magnetic reed switch, Hall effect sensor or other magnetically activated system that actuates temporary changes in the programming of the implantable device. Activation of the magnetic switch minimizes harm to the patient and/or harm to the device which may arise from the EMI exposure.

As shown in FIG. 1, a common approach to activating the magnetic switch in the implantable device to modify or temporarily reprogram the device is to place a magnet 10 over the implanted device 22. Typically, a bulky ceramic ferrite magnet is placed directly on the patient's body 20, such as on the patient's chest, to activate the switch. Removal of the magnet from the patient removes the applied magnetic field and automatically deactivates the switch on the implanted device (i.e. returns the device to its normal mode of operation).

### SUMMARY

In one illustrative embodiment, an adhesive magnetic system for modifying an implantable device is provided. The system includes at least one magnet and an enclosure having an opening, the enclosure configured to receive the at least one magnet through the opening, the enclosure having a first side and a second side. The system also includes an adhesive layer positioned on at least the first side of the enclosure for adhering the enclosure to a patient.

In another illustrative embodiment, an adhesive magnetic system for modifying an implantable device is provided. The system includes at least one non-powered magnet, an enclosure configured to substantially surround the magnet, and an adhesive layer positioned on the enclosure for adhering the enclosure to a patient.

In yet another illustrative embodiment, a method of activating a magnetic switch on a device implanted in a patient is provided. The method includes placing a magnet through an opening within an enclosure such that the enclosure substantially surrounds the magnet, and adhering the enclosure to a patient with an adhesive layer positioned on a first side of the enclosure.

In another embodiment, an adhesive enclosure system for covering a device which is configured to contact a patient is provided. The system includes an enclosure having an opening, the enclosure configured to receive a device through the opening, the enclosure having a first side and a second side, and an adhesive layer positioned on at least the first side of the enclosure for adhering the enclosure to a patient.

Various embodiments of the present invention provide certain advantages. Not all embodiments of the invention share the same advantages and those that do may not share them under all circumstances.

Further features and advantages of the present invention, as well as the structure of various embodiments that incorporate aspects of the invention are described in detail below with reference to the accompanying drawings.

### BRIEF DESCRIPTION OF DRAWINGS

The foregoing and other objects and advantages of the invention will be appreciated more fully from the following drawings, wherein like reference characters designate like features, in which:

FIG. 1 is schematic view of a prior art conventional magnet used to activate a magnetic switch or sensor on an implantable device;

FIG. 2 is a schematic top view of one embodiment of a magnetic system;

FIG. 3 is a schematic side view of the magnetic system shown in FIG. 2;

FIG. 4 is a schematic cross-sectional view of another embodiment of a magnetic system;

FIG. 5 is a schematic perspective view of an embodiment of a magnetic system;

FIG. 6 is a schematic view of one embodiment of a magnetic system positioned on a patient to activate a magnetic switch or sensor on an implantable device;

FIG. 7 is a schematic view of a backing layer on one embodiment of a magnetic system;

FIG. 8 is a schematic view of a magnetic system according to one embodiment;

FIGS. 9-12 are schematic views of magnets according to various embodiments of the magnetic system;

FIG. 13 is a schematic view of a magnet system according to another embodiment;

FIG. 14A-14C illustrate the magnetic field strength between a prior art conventional ceramic magnet and two embodiments of the magnetic system; and

FIG. 15 illustrates the magnetic field profiles for a prior art conventional ceramic magnet and one embodiment of the magnetic system.

### DETAILED DESCRIPTION OF INVENTION

Applicant recognized that there were problems associated with the conventional magnets used to modify an implantable device. First, it may be difficult to securely position the magnet on the patient and ensure that the magnet remains in its required place. This may create risk during a medical procedure, during patient transfer in emergency situations, or any time the patient is transferred or changes position. If the magnet moves away from the implanted device, it could deactivate the magnetic switch or sensor on the implanted device and prematurely cancel the temporary reprogramming of the device. Second, these conventional magnets are typically stored in multiple locations. This may include operating rooms, emergency rooms, procedure rooms, hospital wards, emergency response vehicles, code carts, clinics, and extended care facilities. For convenience, the magnets are often inappropriately attached to objects, such as metal door frames or crash carts, to which they magnetically adhere. Medical staff are often unaware of where they are stored. The magnets are often separated from or do not include instructions for their use. Third, the magnets may be used on multiple patients without protocols for infection control, and are often stored in the open which may expose them to potential pathogens. Applicant recognized that this standard practice could compromise the cleanliness and/or sterility of the magnet and/or the surgical environment. It is known that various bacteria and viruses can spread in healthcare facilities where such surgical procedures typically occur. In particular, there is a concern about Methicillin-Resistant Staphylococcus Aureus (MRSA) infections that are caused by a strain of staph bacteria and are known to occur more often in people who have been in hospitals or other health care settings.

Thus, as described in more detail below, aspects of the present invention are directed to an adhesive magnetic system configured to modify an implantable device. One of ordinary skill in the art would recognize that such a magnetic system may be used to modify the implantable device in a variety of ways, including temporarily reprogramming the implantable device. As discussed above, the implantable device may be configured to be temporary reprogrammed to prevent the device from being harmed by EMI exposure. As discussed below, the magnetic system may be configured to activate the magnetic switch or sensor on an implanted device to temporarily reprogram the device. It should be recognized that the modification or temporary reprogramming of the implantable device may be needed in a variety of circumstances, including, but not limited to surgical or medical procedures. For example, temporary reprogramming to inactivate pacemaker or ICD therapy is often required during the end of a patient's life. Patients often decide they no longer want ICD or pacemaker therapy. Temporary reprogramming with a magnet is often used for this purpose.

Temporary reprogramming is often for an extended period of time and with the patient ambulatory or changing position frequently. Applicant recognized that the current ferrite ceramic magnets are not configured for such use. Accordingly, aspects of the present invention are directed to a magnetic system which includes an adhesive layer to help to place and safely maintain the position of the magnetic on the patient.

Other aspects of the present invention are directed to a magnetic system configured to activate the magnetic switch or sensor on an implanted device, where the magnetic system includes an enclosure configured to surround the magnet. In this respect, the enclosure may shield the patient from direct contact with the magnet, which may help to retain the sterility of the patient's environment.

As set forth below, it is contemplated that the enclosure may be configured for a single use and may be disposable. This would reduce and/or eliminate the risk that multiple patients could come into direct contact with the same enclosure and possibly transmit harmful bacteria or viruses. It is also contemplated that both the enclosure and the magnet may be configured for single use and may be disposable. As set forth below, it is also contemplated that this adhesive enclosure may be used with other devices that are configured to come into direct contact with a patient.

Further aspects of the present invention contemplate a magnet that is less cumbersome than the conventional magnets currently used for temporary reprogramming. As discussed in greater detail below, in one embodiment, rare earth magnets may be used which may have stronger and/or more desirable magnetic properties in comparison to the conventional ceramic ferrite magnets typically used. By using a rare earth magnet, the size of the magnet may be reduced to be less cumbersome, while still providing equal or superior magnetic field properties needed to activate the switch or sensor on the implanted device.

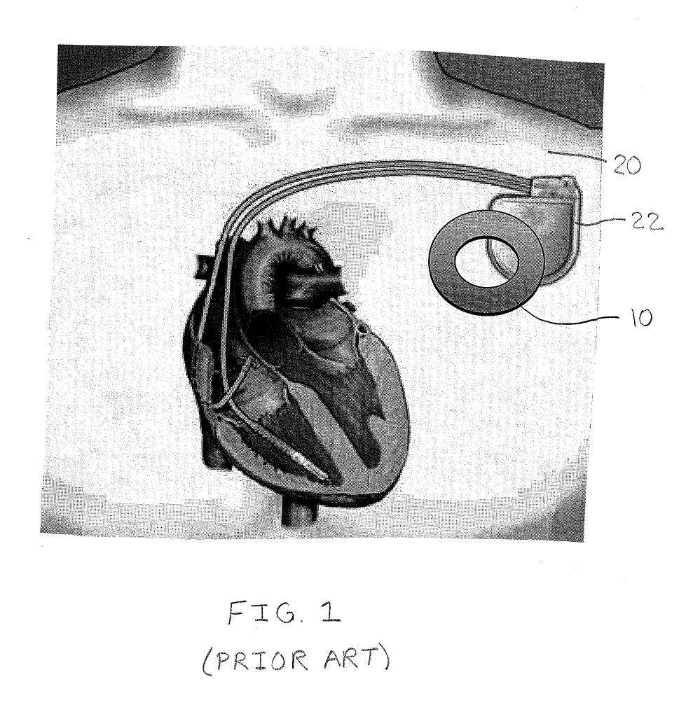

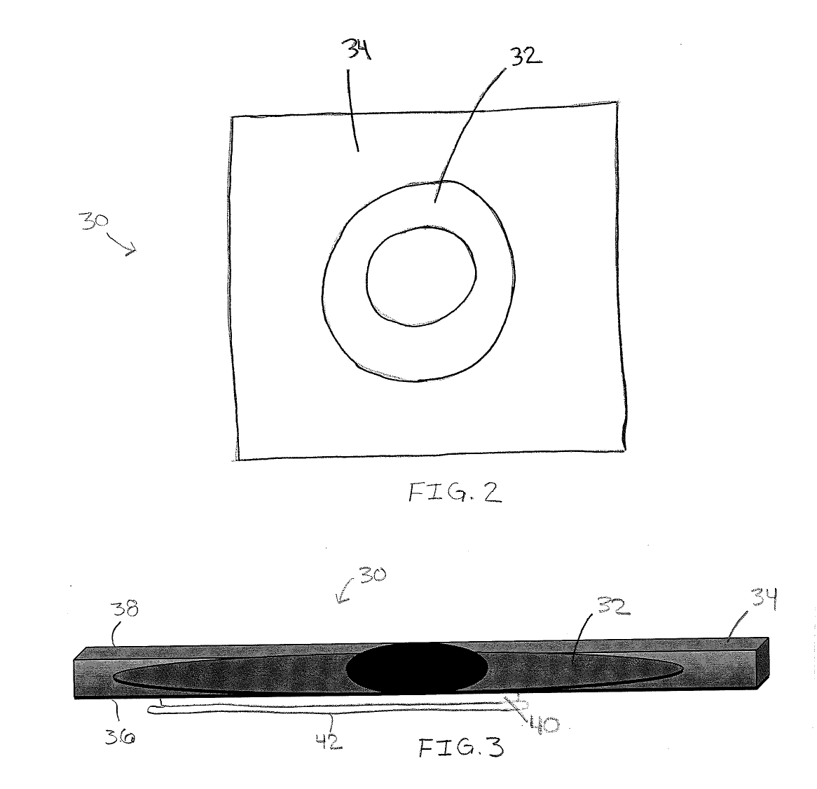

Turning now to FIGS. 2-3, one embodiment of a magnetic system 30 is illustrated. The magnetic system 30 includes a magnet 32 and an enclosure 34 configured to surround the magnet. The enclosure 34 has a first side 36 and a second side 38, and as illustrated, the first side 36 of the enclosure may include an adhesive layer 40 configured for adhering the enclosure to a patient during a modification of a implantable device, such as a temporary reprogramming of a pacemaker or ICD. The system 30 may further include a backing layer 42 coupled to the first side 36 of the enclosure to cover the adhesive layer 40 until the system 30 is adhered to the patient (see also FIG. 7). By including an adhesive layer 40, the magnetic system 30 may be placed on the patient in a desired location over the implanted device and the magnetic system 30 will remain in that position during the modification or reprogramming until it is removed.

It is recognized that during some reprogramming procedures, the patient's body may need to be repositioned. The adhesive layer 40 enables the magnet 32 to remain properly positioned relative to the implantable device. During temporary reprogramming, the patient's body may not be in the supine position with the anterior chest directed upward. Such would be the case in which a patient is having a procedure on their back and is face down or in cases that the patient is positioned on their side. In such circumstances, the adhesive layer 40 enables the magnet 32 to remain properly positioned relative to the implantable device. It is recognized that in some cases, such as during patient transportation in an emergency vehicle, there may be significant movement of the patient which could inadvertently cause movement of the conventional magnet away from the implantable device during temporary reprogramming. The adhesive layer 40 allows the magnet 32 to remain properly positioned relative to the implantable device.

One of ordinary skill in the art would appreciate that a variety of types of adhesive materials may be used to secure the magnetic system 30 to a patient. It may be desirable for the adhesive layer 40 to be strong enough to affix to the patient's skin, and it also may be desirable for the adhesive layer to readily be removed from the patient when the procedure is over. One of ordinary skill in the art would recognize that the adhesive layer may be made of materials such as would be used for medical grade applications designed for prolonged contact with skin.

Turning now to FIG. 4, another embodiment of a magnetic system 50 is illustrated. This embodiment also includes a magnet 32 and an enclosure 34 configured to surround the magnet, where the first side 36 of the enclosure has an adhesive layer 40 configured for adhering the enclosure to a patient. In this embodiment, the enclosure 34 has an opening 60 and the magnet may be insertable through the opening 60, such that the magnet 32 may be removable from the enclosure 34. It is contemplated that the opening 60 may be closable with various types of fasteners and/or seals such as, but not limited to, VELCRO® hook and loop fasteners, zippers, adhesive glues, and slider seals. In one embodiment, the enclosure 34 is configured to be a single use disposable component where the magnet 32 may be removed from the enclosure 34 after the temporary reprogramming and the enclosure 34 may be discarded.

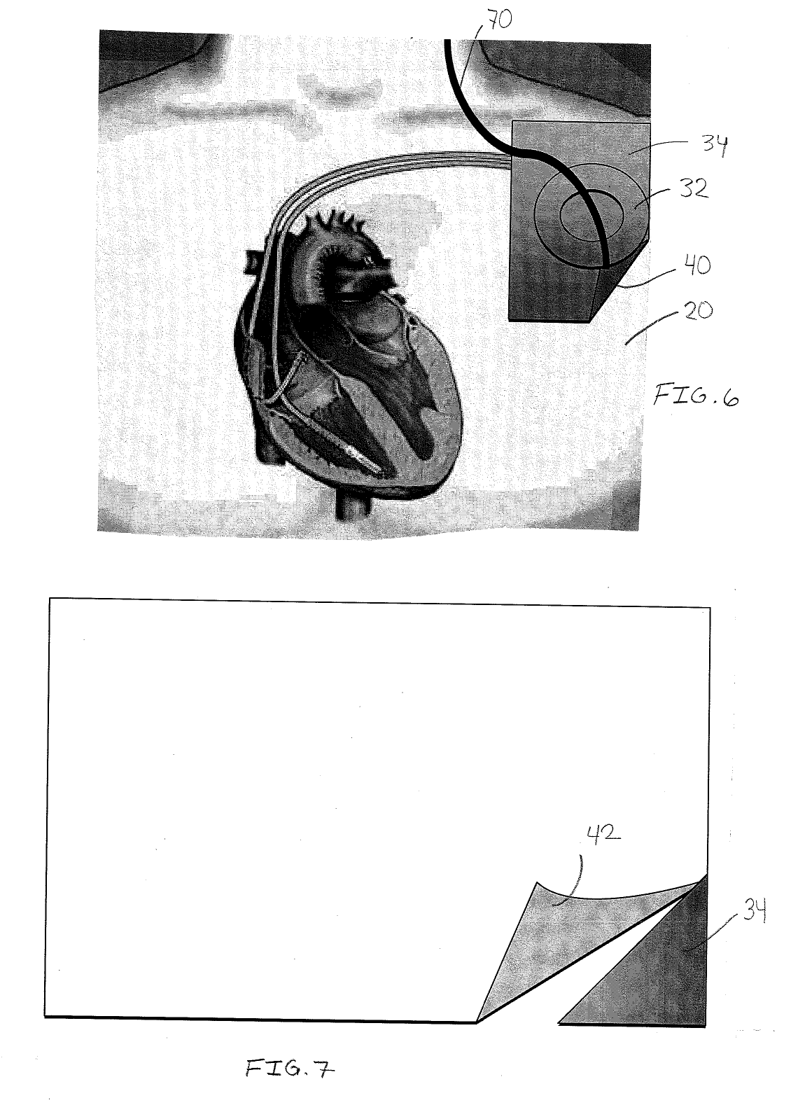

As shown in FIGS. 4-6 and 13, the magnetic system 50, 52, 54 may include a tether 70 coupled to the enclosure 34 configured to facilitate the removal of the enclosure 34 from the patient 20. In other words, the tether 70 may be configured as a pull cord which a caregiver, such as the anesthesiologist, may pull to peel the adhesive layer 40 away from the patient to remove the magnetic system 50 from the patient. Applicant recognized that in some circumstances, it may be desirable and/or necessary to quickly deactivate the switch on the implanted device so that the pacemaker or ICD returns to its normal mode of operation, and such can be accomplished with the tether 70.

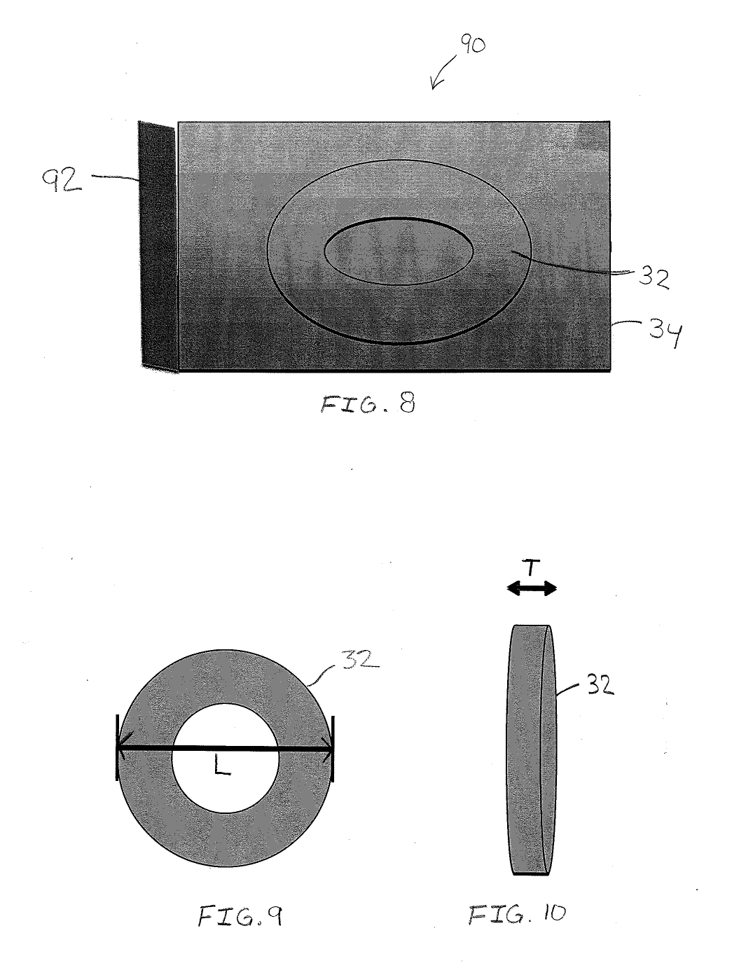

As shown in FIG. 8, a magnetic system 90 may include a tab 92 configured to facilitate the removal of the enclosure 34 from the patient. In this illustrative embodiment, the tab 92 is formed along a side portion of the enclosure 90 and does not include adhesive such that the tab 92 may be pulled to remove the magnetic system 30 from the patient's body. It is also contemplated that one or more tabs 92 may be positioned around the perimeter of the enclosure.

As previously mentioned, the magnet 32 is configured to activate a magnetic switch or sensor (not shown) on a device implanted in a patient 20 when the magnet 32 is placed on the patient during a temporary reprogramming. The magnetic field requirements of the switch, sensor, or other magnetically activated system may be variable between implantable devices. Each switch, sensor, or other magnetically activated system has specific characteristics in terms of being activated by a magnetic field. In one embodiment, the magnetic field of the magnet 32 is at least approximately 40 Gauss measured at an air gap of 2 inches at the outer diameter of the magnet. One of ordinary skill in the art will appreciate that a magnetic field of at least approximately 40 Gauss is typically strong enough to be capable of activating a magnetic switch or sensor in most devices implanted in a patient. In another embodiment, it is also contemplated that the magnet 32 is configured to generate a magnetic field greater than 40 Gauss.

In one embodiment, the magnet 32 is a non-powered permanent magnet that does not rely upon outside influences to generate its magnetic field. In other words, the magnetic properties of the magnet 32 are due to the inherent properties of the material, and the magnet is not an electromagnet material which requires an outside source, such as a power supply, to create a current to generate the magnetic field.

A conventional ceramic ferrite magnet used to activate an implanted switch may be cumbersome due to the size, weight and thickness of the magnet needed to achieve the desired magnetic field strength and/or properties. Thus, according to one aspect of the present invention, alternative materials, which have stronger and/or more desirable magnetic properties, are used to form the magnet 32. For example, in one embodiment, a rare earth magnet is used. Rare earth magnets are made from substances that hold a more dense and/or powerful magnetic flux in comparison to regular magnets, such as ceramic ferrite magnets. By using a rare earth magnet, the dimensions of the magnet and the mass of the magnet may be reduced to be less cumbersome, while still providing the minimum magnetic properties needed to safely activate the switch or sensor on the implanted device. For example, in one embodiment, the magnet 32 may be made with a rare earth magnet known as Neodymium. Other rare earth magnets, such as, but not limited to Samarium cobalt may also be used. In one embodiment, the magnet 32 is made of Neodymium and may be obtained from a commercial magnet manufacturer, such as Dexter Magnetic Technologies. Multiple grades and composites or alloys of magnetic material are available and may be used. It is contemplated that the magnet may be either rigid or flexible. The external surfaces of the magnet may be coated with a separate material such as nickel plating or other such material to provide appropriate protection from oxidation of the magnetic material.

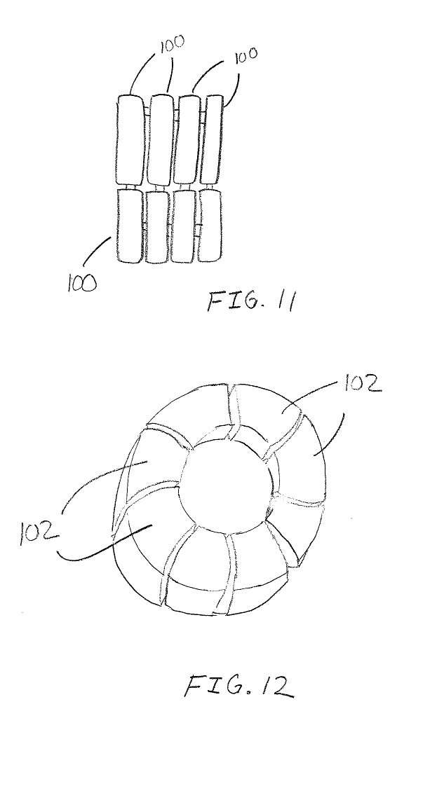

As shown in FIGS. 9-10, in one embodiment, the magnet 32 is substantially toroid shaped. It should also be appreciated that other shaped magnets 32 are also contemplated, such as, but not limited to square shaped, triangular shaped, heart shaped, rectangular shaped, and irregular shaped magnets.

Applicant recognized that it would be desirable to have a magnet 32 that is less cumbersome than the conventional ferrite ceramic magnet 10. It is contemplated that a thinner, lighter, and/or smaller magnet would more readily remain in its desired position on the patient's body during temporary reprogramming. In one embodiment, the thickness T of the magnet 32 is approximately 0.22 inches. In another embodiment, the thickness T of the magnet 32 is approximately 0.30 inches, and in another embodiment, the thickness of the magnet 32 is approximately 0.5 inches. This is in contrast to a conventional ceramic magnet 10 which is currently used for temporary reprogramming that has a thickness of at least approximately 0.59 inches.

In one embodiment, the maximum outer diameter L of the magnet 32 is less than approximately 3 inches. In another embodiment, the maximum outer diameter L of the magnet 32 is approximately 3.5 inches, and in another embodiment, the outer diameter L of the magnet 32 is less than approximately 4.0 inches. A conventional ceramic magnet 10 that is currently used for temporary reprogramming has an outer diameter of approximately 3 inches.

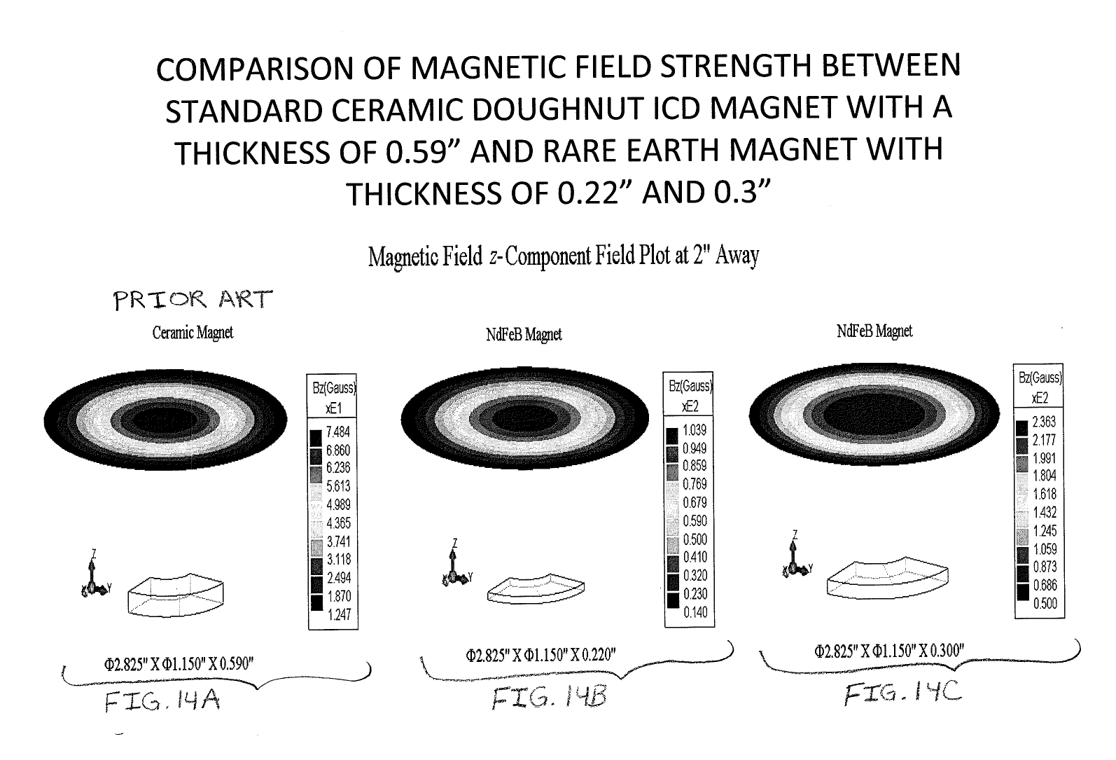

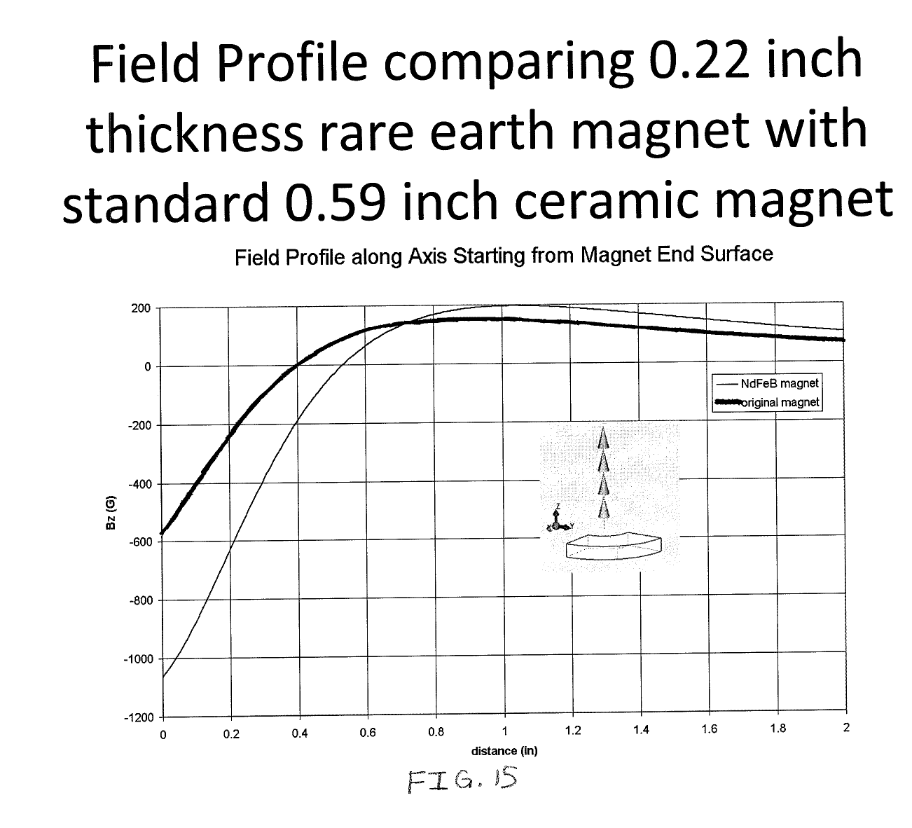

FIGS. 14A-C illustrate the magnetic field strength between a prior art conventional ceramic magnet and two embodiments of the magnetic system according to the present invention. The three magnets are each substantially toroid shaped and have substantially the same maximum outer diameter of approximately 2.825 inches and an inside diameter of approximately 1.15 inches, although each have differing thicknesses. The ceramic magnet shown in FIG. 14A has a thickness of 0.59 inches. As shown in FIG. 14A, at approximately 2 inches away from the magnet, the prior art ceramic magnet has a maximum magnetic field strength of approximately 75 Gauss (note that the scale of the chart is xE1 which is ×10). The magnetic field strength is the highest at the center of the magnet and as shown, is reduced in a radial direction. FIG. 14B illustrates the magnetic field strength for a rare earth magnet 32 made of Neodymium according to one embodiment of the present invention which has a thickness of only 0.22 inches. As shown in FIG. 14B, at approximately 2 inches away from the magnet, this magnet 32 has a maximum magnetic field strength of approximately 104 Gauss (note the scale of this chart is xE2, which is ×100). The magnetic field strength is the highest at the center of the magnet and as shown, is reduced in a radial direction. FIG. 14C illustrates the magnetic field strength for a rare earth magnet 32 made of Neodymium according to another embodiment of the present invention which has a thickness of only 0.30 inches. As shown in FIG. 14C, at approximately 2 inches away from the magnet, this magnet 32 has a maximum magnetic field strength of approximately 236 Gauss (note the scale of this chart is xE2, which is ×100).

FIG. 15 illustrates the magnetic field profiles for a prior art conventional ceramic magnet and one embodiment of the magnetic system. In particular, this graph compares the conventional ceramic magnet shown in FIG. 14A with the Neodymium magnet shown in FIG. 14B. All dimensions of the two magnets are substantially identical except for the thickness. The thickness of the ceramic magnet is approximately 0.59 inches while the thickness of the Neodymium magnet is only approximately 0.22 inches. As shown, despite the rare earth magnet being thinner, the magnetic field strength of the rare earth magnet is substantially larger than the conventional ceramic magnet at distances greater than 0.8 inches.

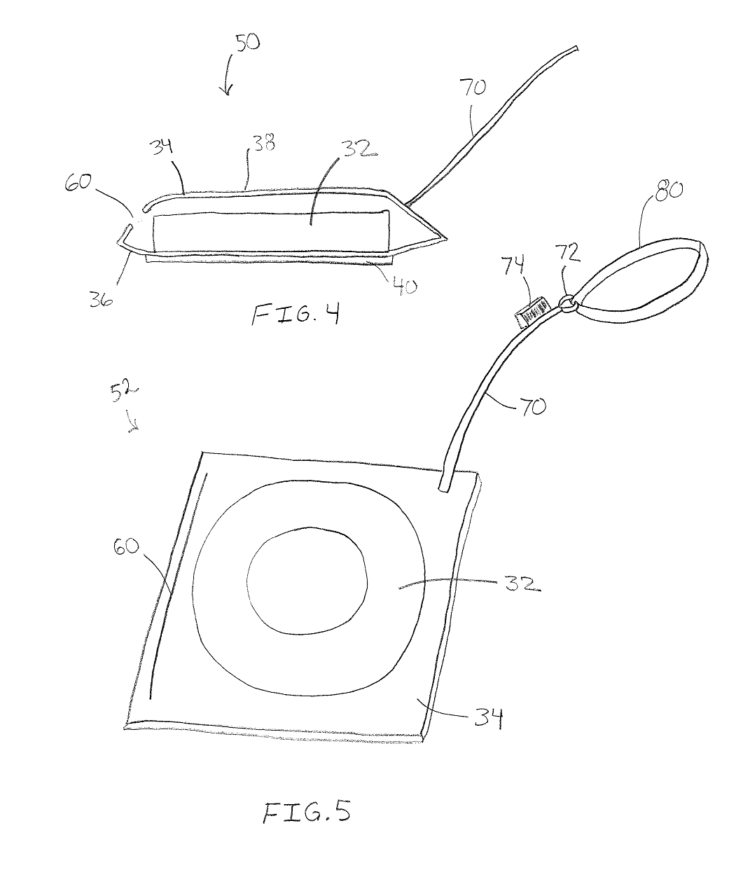

The present invention also contemplates physically linking the magnetic system 30, 50, 52, 54 to the patient to ensure that the magnetic system remains with the patient such as before, during and/or after a surgical procedure. This may also act as a reminder or indicator that the patient has a magnet in place and that it may need to be removed to discontinue temporary reprogramming. This may help to reduce the likelihood that the magnetic system is used with multiple patients. This may also help to ensure that the magnetic system is easily accessible when needed during a surgical procedure. As shown in FIGS. 5 and 13, the tether 70 may be coupled to a bracelet 80 which may be configured to be worn by the patient 20. As shown in FIG. 5, the tether 70 may be coupled to the bracelet via a connector 72. It is contemplated that the tether 70, connector 72 and bracelet 80 may be configured to physically link the magnetic system 30 to the patient before the magnetic system is adhered to the patient during a temporary reprogramming, such as, but not limited to during a surgical procedure. In other words, the tether 70, bracelet 80 and connector 72 may be configured to couple the magnetic system 52, 54 to the patient without affecting the magnetic switch on any device implanted in the patient. As shown in FIG. 7, a barcode 74 may also be provided on the tether 70 or bracelet 80 which may be used to track the magnet use on the particular patient. Also, as shown in FIG. 13, a tab 76 on the enclosure 34 may couple the tether 70 to the enclosure 34.

Applicant recognized that it may be desirable to cover the magnet 32 in an enclosure 34. In this respect, the enclosure 34 may shield the patient from direct contact with the magnet. This may help to retain the sterility and/or hygiene of the patient's environment, be more comfortable, and avoid injury to the patient with prolonged requirements for temporary reprogramming. In one embodiment, the enclosure is made of a medical grade material, such as, but not limited to commercially available films, tapes, and fabrics. In one embodiment, the enclosure is made of a substantially impermeable material which substantially prevents the passage of viruses or bacteria therethrough. In another embodiment the material contains an embedded or impregnated bacteriostatic or bactericidal component.

In one embodiment, the enclosure is configured as a pouch or bag, and may for example, be made of a substantially flexible plastic, or other synthetic sheet material. In the embodiment illustrated in FIG. 13, the enclosure 34 has an envelope-like configuration. In one embodiment, the enclosure is made of a substantially transparent material. It is contemplated that transparent portions of the enclosure may be used to place the magnet 30 positioned within the enclosure 34 on the patient 20 to align with the implanted device.

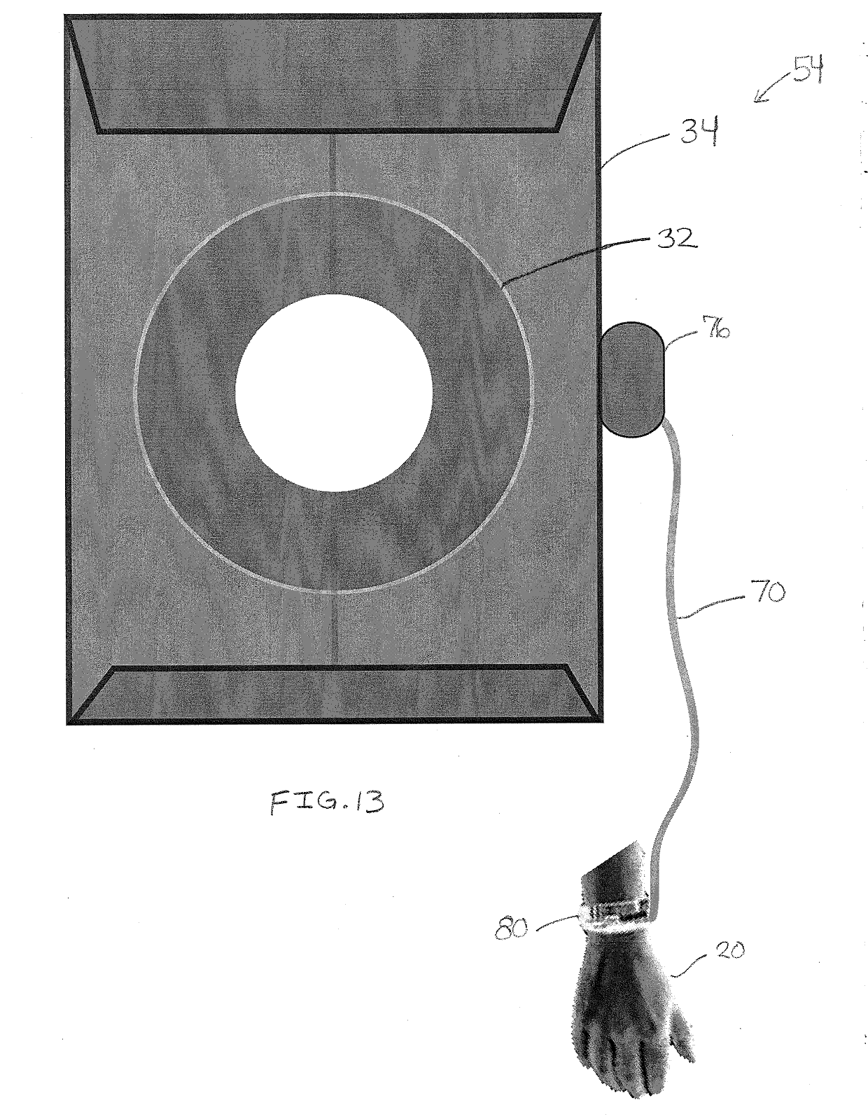

Applicant recognized that there are difficulties associated with placing and retaining a conventional magnet 10 on the patient. Applicant further recognized that a plurality of smaller magnets may be easier to position on a patient. For example, FIGS. 11-12 illustrate two additional embodiments of magnet configurations. In particular, in FIG. 11, a plurality of magnets 100 are provided and in this embodiment, the plurality of magnets 100 are coupled together. The magnets 100 may be coupled together in a manner which provides some movement between adjacent magnets. This may enable the magnet to adjust to the contour of the patient's body. FIG. 12 illustrates another configuration of a plurality of magnets 102. In this embodiment, the plurality of magnets 102 together are substantially toroid shaped. As mentioned above, other magnet shapes and configurations are also contemplated as the invention is not so limited. The plurality of magnets 100, 102 may be placed within an enclosure 34 for use during a modification of an implantable device.

It is contemplated that the enclosure 34 may be configured for a single use and may be disposable. This would reduce and/or eliminate the risk that multiple patients could come into direct contact with the same enclosure and possibly transmit harmful bacteria or viruses. The magnet 30 may be reused with multiple patients, but because the enclosure 34 shields the patient from direct contact with the magnet 30, the risk of compromising the sterility and/or hygiene of the patient's environment is minimized. It is also contemplated that the combination of both the enclosure and the magnet may be configured for single use and may be disposable. It is also contemplated that the material making up the magnet and enclosure would be suitable for sterilization.

Furthermore, Applicant also recognized that the above described enclosure 34 may also be used with devices, other than a magnet system, which are configured to directly contact a patient. For example, it is contemplated that such an adhesive enclosure system may be configured for use with a fetal heart monitor. The adhesive layer on the enclosure enables the enclosure to be adhered to a patient and the enclosure is configured to cover or shield the device.

It should be appreciated that various embodiments of the present invention may be formed with one or more of the above-described features. The above aspects and features of the invention may be employed in any suitable combination as the present invention is not limited in this respect. It should also be appreciated that the drawings illustrate various components and features which may be incorporated into various embodiments of the present invention. For simplification, some of the drawings may illustrate more than one optional feature or component. However, the present invention is not limited to the specific embodiments disclosed in the drawings. It should be recognized that the present invention encompasses embodiments which may include only a portion of the components illustrated in any one drawing figure, and/or may also encompass embodiments combining components illustrated in multiple different drawing figures.

It should be understood that the foregoing description of various embodiments of the invention are intended merely to be illustrative thereof and that other embodiments, modifications, and equivalents of the invention are within the scope of the invention recited in the claims appended hereto.

What is claimed is:

1. An adhesive magnetic system for modifying an implantable device, the system comprising:
at least one magnet;
an enclosure having an opening, the enclosure configured to receive the at least one magnet through the opening, the enclosure having a first side and a second side; and
an adhesive layer positioned on at least the first side of the enclosure for adhering the enclosure to a patient.

2. The adhesive magnetic system of claim 1, further comprising:
a backing layer coupled to the first side of the enclosure, the backing layer configured to cover the adhesive layer.

3. The adhesive magnetic system of claim 1, further comprising a tether coupled to the enclosure, the tether being configured to facilitate removal of the enclosure from the patient.

4. The adhesive magnetic system of claim 1, wherein the at least one magnet is substantially toroid shaped.

5. The adhesive magnetic system of claim 1, wherein the at least one magnet is a permanent, non-powered magnet.

6. The adhesive magnetic system of claim 1, wherein the thickness of the at least one magnet is less than 0.59 inches.

7. The adhesive magnetic system of claim 1, wherein the at least one magnet includes a plurality of magnets.

8. The adhesive magnetic system of claim 7, wherein the plurality of magnets together are substantially toroid shaped.

9. The adhesive magnetic system of claim 1, wherein the enclosure is made of a substantially impermeable material.

10. The adhesive magnetic system of claim 1, wherein the enclosure is made of a substantially flexible plastic sheet material.

11. The adhesive magnetic system of claim 1, wherein the enclosure is made of a substantially transparent material.

12. An adhesive magnetic system for modifying an implantable device, the system comprising:
at least one non-powered magnet;
an enclosure configured to substantially surround the magnet; and
an adhesive layer positioned on the enclosure for adhering the enclosure to a patient.

13. The adhesive magnetic system of claim 12, wherein the enclosure includes an opening constructed and arranged such that the at least one magnet is removable from the enclosure through the opening.

14. The adhesive magnetic system of claim 12, further comprising:
a backing layer coupled to the enclosure, the backing layer configured to cover the adhesive layer.

15. The adhesive magnetic system of claim 12, further comprising a tether coupled to the enclosure, the tether being configured to facilitate removal of the enclosure from the patient.

16. The adhesive magnetic system of claim 12, wherein the at least one magnet is substantially toroid shaped.

17. The adhesive magnetic system of claim 12, wherein the at least one magnet includes a plurality of magnets.

18. The adhesive magnetic system of claim 12, wherein the enclosure is made of a substantially impermeable material.

19. The adhesive magnetic system of claim 12, wherein the enclosure is made of a substantially flexible plastic sheet material.

20. A method of activating a magnetic switch on a device implanted in a patient, the method comprising:
placing a magnet through an opening within an enclosure such that the enclosure substantially surrounds the magnet; and
adhering the enclosure to a patient with an adhesive layer positioned on a first side of the enclosure.

21. The method of claim 20, further comprising:
pulling a tether coupled to the enclosure to facilitate removal of the enclosure from the patient.

22. An adhesive enclosure system for covering a device which is configured to contact a patient, the system comprising:
an enclosure having an opening, the enclosure configured to receive a device through the opening, the enclosure having a first side and a second side; and
an adhesive layer positioned on at least the first side of the enclosure for adhering the enclosure to a patient.

23. The adhesive enclosure system of claim 22, in combination with the device which is configured to contact a patient.

24. The adhesive enclosure system of claim 23, wherein the device is at least one magnet.

25. The adhesive enclosure system of claim 23, wherein the device is a fetal heart monitor.
